(19) United States
(12) Patent Application Publication
(10) Pub. No.: US 2013/0288236 A1
(43) Pub. Date: Oct. 31, 2013

(54) WT1 MUTATIONS FOR PROGNOSIS OF MYELOPROLIFERATIVE DISORDERS

(71) Applicants: Maher Albitar, Coto De Caza, CA (US); Wanlong Ma, Aliso Viejo, CA (US)

(72) Inventors: Maher Albitar, Coto De Caza, CA (US); Wanlong Ma, Aliso Viejo, CA (US)

(73) Assignee: Quest Diagnostics Investments Incorporated

(21) Appl. No.: 13/509,271

(22) PCT Filed: Oct. 29, 2010
(86) PCT No.: PCT/US10/54830

§ 371 Date: Aug. 31, 2012

Related U.S. Application Data
(60) Provisional application No. 61/260,344, filed on Nov. 11, 2009.

(51) Int. Cl. C12Q 1/68

(52) U.S. Cl. CPC C12Q 1/6886
USPC 435/6.11

(57) ABSTRACT

The invention provides methods for determining the prognosis of a patient diagnosed with a leukemia, including B-cell chronic lymphocytic leukemia, by measuring mutations of the WT1 gene in a biological sample. The invention also relates to the diagnosis of leukemia, including B-cell chronic lymphocytic leukemia.

### TECHNICAL FIELD

The present invention relates generally to the field of cancer diagnostics and, in particular, the diagnosis and prognosis of patients having leukemia.

### BACKGROUND

The following description is provided to assist the understanding of the reader. None of the information provided or references cited is admitted to be prior art to the present invention.

Cancer describes a class of disorders and diseases characterized by the uncontrolled growth of aberrant cells. Currently, cancer is one of the most deadly diseases with about 1.2 million new cases of cancer being diagnosed each year in the United States of America alone.

One form of cancer, accounting for about 3% of all cancers in the United States of America, is leukemia. This malignant disease is characterized by an abnormal proliferation of white blood cells which can be detected in the peripheral blood and/or bone marrow. Leukemia can be broadly classified into acute and chronic leukemia. Further classification within these groups is essential as a precise diagnosis is necessary in order to determine prognosis and guide the choice of treatment. The acute leukemias can be subclassified into myeloid and lymphoid leukemias in a variety of ways, including cell morphology and cytochemistry. Despite the obvious morphological differences between the myeloid and lymphoid leukemias, immunophenotyping and cytogenetic analysis have become increasingly important in recent years to confirm and/or supplement diagnosis of leukemias.

Acute myeloid leukemia (AML) is the most common form of leukemia accounting for about 50% of all leukemia cases and even 85% of all acute leukemia cases involving adults. As a general criterion for the classification as AML, the cytoplasmic antigen myeloperoxidase should be demonstrated in the abnormal cell population either cytochemically or immunologically. Furthermore, the diagnosis of AML is supported by the expression of lineage-associated markers. Only a limited number of markers commonly expressed by a large number of AML-subtypes, such as CD13 and CD33, have been identified.

B-cell chronic lymphocytic leukemia (B-CLL) is characterized by a progressive accumulation of long-lived and well-differentiated clonal B-lymphocytes. Although B-CLL pathogenesis is not entirely understood, the progressive increase in lymphocyte counts coupled with the very low proportion of proliferating cells suggests that B-CLL may be primarily driven by defective apoptosis. B-CLL has a highly varied course in affected subjects. B-CLL can be categorized in at least two different subgroups, with different clinical outcome. See, e.g. Wiestner, A., Rosenwald, A., Barry, T. S., Wright, G., et al., Blood, 101:4944-4951 (2003); Hamblin, T., Ann Hematol 2002, 81: 99-303 (2002); Rozman, C. and Montserrat, E., N Engl J Med, 333:1052-1057 (1995); Chen, L., Widhopf, G., Huynh, L., Rassenti, L., et al., Blood, 100:4609-4614 (2002).

Some patients have an indolent disease with little need for therapeutic intervention, while other patients show a more aggressive clinical course requiring therapeutic intervention. Less aggressive forms of B-CLL are generally monitored but not treated since the risks associated with therapy can outweigh the benefits. However, other forms of the disease can progress rapidly resulting in uncomfortable symptoms, repeated serious infections and death, warranting aggressive therapeutic intervention such as bone marrow transplant, chemotherapy and monoclonal antibody therapy. The significant differences in clinical outcome associated with diverse forms of B-CLL make it especially important to be able to distinguish more aggressive forms of the disease from less aggressive forms.

The Wilms' Tumor (WT1) gene was isolated from chromosome 11p13 by a positional cloning technique (Haber et al., Cell 1990, Vol. 61, pgs 1257-1269) and consists of 10 exons. The WT1 gene encodes a transcriptional regulator with an N-terminal domain (exons 1 to 6) and a C-terminal domain (exons 7 to 10). WT1 has been observed to have dual functionality; behave both as a tumor suppressor gene and an oncogene. The WT1 gene is highly expressed in 70% to 90% of patients with AML, thus is a useful marker for monitoring minimal residual disease.

### SUMMARY OF THE INVENTION

The present invention is based on the identification of a single nucleotide polymorphism in the WT1 gene that affects the prognosis of patients diagnosed as having a myeloproliferative disease. Specifically, a single base alteration of an adenine to a guanine to at nucleotide position 903 of SEQ ID NO: 1 (cDNA sequence of the WT1 gene) was identified in heterozygous (A/G) and homozygous wild type (A/A) patients who had inferior clinical outcomes relative to those patients who were homozygous mutant (G/G).

In one aspect, the invention provides a method of determining a prognosis for a subject diagnosed with a myeloproliferative disease by determining the zygosity status of the subject at the nucleotide corresponding to position 903 of SEQ ID NO: 1; and identifying the subject as having a poor prognosis when the subject has the A/G or A/A genotype at position 903. For convenience, the nucleotide position is referred to as the wildtype A903, but it is recognized that a determination of the zygosity status/genotype involves determining the identity of the nucleotide at that position which will not necessarily be an adenine. In some embodiments, the subject has been diagnosed as having chronic lymphocytic leukemia, acute lymphoblastic leukemia, acute myelogenous leukemia, chronic myelogenous leukemia, hairy cell leukemia, T-cell prolymphocytic leukemia, large granular lymphocytic leukemia. Individuals assessed as having the A/G or A/A genotypes are identified as having a poor prognosis relative to individuals having the G/G genotype. A poor prognosis may be expressed as shorter survival, shorter complete remission duration, or reduced probability of an event-free survival. Optionally, the prognosis is made in further view of one or more clinical factors including, for example, cytogenetics, peripheral white blood cell count, percentage of blast cells in bone marrow, and percentage of blast cells in blood. In other preferred embodiments, the subjects are less than 50 years of age. In some embodiments, the invention also comprises assessing the presence or absence of a FLT3 mutation and identifying the subject as having a poor prognosis when a FLT3 mutation is present. The zygosity status of the WT1 gene may be assessed in any convenient nucleic acid type derived from a biological sample. Suitable nucleic acids include genomic DNA and RNA including mRNA, and cDNA that has been reverse transcribed from RNA. Biological samples include any convenient cellular or acellular body fluid, isolated blood cells, and tissue samples (e.g., biopsy specimens). In some embodiments, the zygosity status is determined using a technique selected from the group consisting of nucleic acid sequencing, probe hybridization, and primer extension reaction.

In another aspect, the invention provides a method of determining a prognosis for a subject diagnosed as having a myeloproliferative disease by a) determining the zygosity status of the subject for at least one of the mutations in the group comprising 902-939 dup, 929-933 dup, 939-959 dup, C938A, 912-917 del, 912-917indel14, 912-917indel23; and b) identifying the subject as having a poor prognosis when the subject is heterozygous or homozygous for the mutation. In some embodiments, the mutation assessed is 902-939 dup, 929-933 dup, or 939-959 dup. In other embodiments, the mutation assessed is C938A. In still other embodiments, the mutation assessed is 912-917 del, 912-917indel14, 912-917indel23. In some embodiments, the subject has been diagnosed as having chronic lymphocytic leukemia, acute lymphoblastic leukemia, acute myelogenous leukemia, chronic myelogenous leukemia, hairy cell leukemia, T-cell prolymphocytic leukemia, large granular lymphocytic leukemia. Individuals assessed as having the heterozygous and homozygous mutations are identified as having a poor prognosis relative to individuals having homozygous wild type genotype. A poor prognosis may be expressed as shorter survival, shorter complete remission duration, or reduced probability of an event-free survival. Optionally, the prognosis is made in further view of one or more clinical factors including, for example, cytogenetics, peripheral white blood cell count, percentage of blast cells in bone marrow, and percentage of blast cells in blood. In other preferred embodiments, the subjects are less than 50 years of age. In some embodiments, the invention also comprises assessing the presence or absence of a FLT3 mutation and identifying the subject as having a poor prognosis when a FLT3 mutation is present.

In yet another aspect, the invention provides a method of determining the myeloproliferative disease status of a subject by: a) determining the zygosity status of the subject for at least one of the mutations of the WT 1 gene selected from the group consisting of 902-939 dup, 929-933 dup, 939-959 dup, C938A, 912-917 del, 912-917indel14, and 912-917indel23; and b) identifying the subject i) as having a myeloproliferative disease when the subject is homozygous for one or more of said WT1 mutations, ii) as being predisposed to a myeloproliferative disease when the subject is heterozygous for one or more of said WT1 mutations, or iii) as having no predisposition caused by one of said WT1 mutations when said WT1 mutations are absent from both alleles of the WT1 gene. In some embodiments, the mutation assessed is 902-939 dup, 929-933 dup, or 939-959 dup. In other embodiments, the mutation assessed is C938A. In still other embodiments, the mutation assessed is 912-917 del, 912-917indel14, 912-917indel23. In some embodiments, the subject is diagnosed as having chronic lymphocytic leukemia, acute lymphoblastic leukemia, acute myelogenous leukemia, chronic myelogenous leukemia, hairy cell leukemia, T-cell prolymphocytic leukemia, large granular lymphocytic leukemia. In other preferred embodiments, the subjects are less than 50 years of age. In some embodiments, the invention also comprises assessing the presence or absence of a FLT3 mutation and identifying the subject as having a poor prognosis when a FLT3 mutation is present.

In still another aspect, the invention provides oligonucleotide primers and probes, and diagnostic kits containing the same, for assessing the zygosity status of A903G, C938A, 902-939 dup, 929-933 dup, 939-959 dup, 912-917 del, 912-917indel14, and 912-917indel23. In one embodiment, the kit contains amplification primers that are specific for the WT1 gene or fragment thereof and flank the SNPs and mutations. Preferably the primers amplify a nucleic acid fragment of at least about 30, 40, 50, 60, 70, 80, 100, 125, 150, 200, 300, 400, 500, or more nucleotides of the WT1 gene. The kit may further contain an oligonucleotide probe that specifically hybridizes to either the G903 or A903 polymorphism. Optionally, the probe is detectably labeled or contains a binding pair member to facilitate the attachment of a detectable label. In another embodiment, the kit contains at least one an allele-specific amplification primer that is capable of specifically hybridizing to either the G903 or A903 polymorphism. Typically, this kit will also contain a reverse primer. The allele-specific amplification primer optionally may contain a detectable label. In another embodiment, the kit contains an oligonucleotide capable of performing a primer extension reaction and one or more detectably-labeled nucleotides. Preferably, the 3′ terminal of the primer corresponds and is complementary to nucleotide position 903 of SEQ ID NO: 1 and the nucleotides are incapable of further chain extension (e.g., ddNTPs).

The term “myeloproliferative disease” as used herein means a disorder of a bone marrow or lymph node-derived cell type, such as a white blood cell. A myeloproliferative disease is generally manifest by abnormal cell division resulting in an abnormal level of a particular hematological cell population. The abnormal cell division underlying a proliferative hematological disorder is typically inherent in the cells and not a normal physiological response to infection or inflammation. Leukemia is a type of myeloproliferative disease. Exemplary myeloproliferative diseases include, but are not limited to, acute myeloid leukemia (AML), acute lymphoblastic leukemia (ALL), chronic lymphocytic leukemia (CLL), myelodysplastic syndrome (MDS), chronic myeloid leukemia (CML), hairy cell leukemia, leukemic manifestations of lymphomas, multiple myeloma, polycythemia vera (PV), essential thrombocythemia (ET), idiopathic myelofibrosis (IMF), hypereosinophilic syndrome (HES), chronic neutrophilic leukemia (CNL), myelofibrosis with myeloid metaplasia (MMM), chronic myelomonocytic leukemia (CMML), juvenile myelomonocytic leukemia, chronic basophilic leukemia, chronic eosinophilic leukemia, systemic mastocytosis (SM), and unclassified myeloproliferative diseases (UMPD or MPD-NC). Lymphoma is a type of proliferative disease that mainly involves lymphoid organs, such as lymph nodes, liver, and spleen. Exemplary proliferative lymphoid disorders include lymphocytic lymphoma (also called chronic lymphocytic leukemia), follicular lymphoma, large cell lymphoma, Burkitt's lymphoma, marginal zone lymphoma, lymphoblastic lymphoma (also called acute lymphoblastic lymphoma).

The term “diagnose” or “diagnosis” or “diagnosing” as used herein refer to distinguishing or identifying a disease, syndrome or condition or distinguishing or identifying a person having a particular disease, syndrome or condition. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease. In this regard, the term means assessing whether or not an individual or a subject has a mutation in the WT1 gene.

The term “prognosis” as used herein refers to a prediction of the probable course and outcome of a clinical condition or disease. A prognosis of a patient is usually made by evaluating factors or symptoms of a disease that are indicative of a favorable or unfavorable course or outcome of the disease.

The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. A prognosis may be expressed as the amount of time a patient can be expected to survive. Alternatively, a prognosis may refer to the likelihood that the disease goes into remission or to the amount of time the disease can be expected to remain in remission. Prognosis can be expressed in various ways; for example prognosis can be expressed as a percent chance that a patient will survive after one year, five years, ten years or the like. Alternatively prognosis may be expressed as the number of years, on average that a patient can expect to survive as a result of a condition or disease. The prognosis of a patient may be considered as an expression of relativism, with many factors effecting the ultimate outcome. For example, for patients with certain conditions, prognosis can be appropriately expressed as the likelihood that a condition may be treatable or curable, or the likelihood that a disease will go into remission, whereas for patients with more severe conditions prognosis may be more appropriately expressed as likelihood of survival for a specified period of time.

The term “poor prognosis” as used herein, in the context of a patient having a leukemia and the A/G or A/A R301 genotype, refers to an increased likelihood that the patient will have a worse outcome in a clinical condition relative to a patient diagnosed as having the same disease but having the G/G R301 genotype. A poor prognosis may be expressed in any relevant prognostic terms and may include, for example, the expectation of a reduced duration of remission, reduced survival rate, and reduced survival duration.

The term “zygosity status” as used herein refers to a sample, a cell population, or an organism as appearing heterozygous, homozygous, or hemizygous as determined by testing methods known in the art and described herein. The term “zygosity status of a nucleic acid” means determining whether the source of nucleic acid appears heterozygous, homozygous, or hemizygous. The “zygosity status” may refer to differences in a single nucleotide in a sequence. In some methods, the zygosity status of a sample with respect to a single mutation may be categorized as homozygous wild-type, heterozygous (i.e., one wild-type allele and one mutant allele), homozygous mutant, or hemizygous (i.e., a single copy of either the wild-type or mutant allele). For example, the zygosity status identifies whether an individual has the A/A, A/G, or G/G genotype for the WT1 gene at the nucleotide position corresponding to position 903 of SEQ ID NO: 1.

The zygosity status in a sample may be determined by methods known in the art including sequence-specific, quantitative detection methods. Other methods may involve determining the area under the curves of the sequencing peaks from standard sequencing electropherograms, such as those created using ABI Sequencing Systems, (Applied Biosystems, Foster City Calif.). For example, the presence of only a single peak such as a “G” on an electropherogram in a position representative of a particular nucleotide is an indication that the nucleic acids in the sample contain only one nucleotide at that position, the “G.” The sample may then be categorized as homozygous because only one allele is detected. The presence of two peaks, for example, a “G” peak and an “A” peak in the same position on the electropherogram indicates that the sample contains two species of nucleic acids; one species carries the “G” at the nucleotide position in question, the other carries the “A” at the nucleotide position in question. The sample may then be categorized as heterozygous because more than one allele is detected.

As used herein, the term “predisposed” or “predisposition” refers to an increased likelihood that a patient may be afflicted with a disease. A single mutation on a single allele of a gene may not be sufficient to lead to a disease state in an individual. However, different disease-causing mutations on separate alleles could lead to disease. For example, if a person is heterozygous for a single WT1 mutation on a single allele, the remaining wildtype allele may be enough to prevent myeloproliferative disease in that person. However, if that person subsequently acquires a different WT1 mutation on the remaining wildtype WT1 allele, they may then be stricken with a myeloproliferative disease. Thus, a person with one mutation on a single allele of WT1 has an increased likelihood of being afflicted with a myeloproliferative disease because only one wildtype allele of WT1 remains.

As used herein, the term “sample” or “biological sample” refers to any liquid or solid material obtained from a biological source, such a cell or tissue sample or bodily fluids. “Bodily fluids” include, but are not limited to, blood, serum, plasma, saliva, cerebrospinal fluid, pleural fluid, tears, lactal duct fluid, lymph, sputum, urine, saliva, amniotic fluid, and semen. A sample may include a bodily fluid that is “acellular.” An “acellular bodily fluid” includes less than about 1% (w/w) whole cellular material. Plasma or serum are examples of acellular bodily fluids. A sample may include a specimen of natural or synthetic origin. Exemplary sample tissues include, but are not limited to bone marrow or tissue (e.g. biopsy material).

As used herein, the term “specifically binds,” when referring to a binding moiety, is meant that the moiety is capable of discriminating between a various target sequences. For example, an oligonucleotide (e.g., a primer or probe) that specifically binds to a mutant target sequence is one that hybridizes preferentially to the target sequence (e.g., the wildtype sequence) over the other sequence variants (e.g., mutant and polymorphic sequences). Preferably, oligonucleotides specifically bind to their target sequences under high stringency hybridization conditions.

As used herein the term “stringency” is used in reference to the conditions of temperature, ionic strength, and the presence of other compounds, under which nucleic acid hybridizations are conducted. With high stringency conditions, nucleic acid base pairing will occur only between nucleic acids that have sufficiently long segment with a high frequency of complementary base sequences.

Exemplary hybridization conditions are as follows. High stringency generally refers to conditions that permit hybridization of only those nucleic acid sequences that form stable hybrids in 0.018M NaCl at 65° C. High stringency conditions can be provided, for example, by hybridization in 50% formamide, 5×Denhardt's solution, 5×SSC (saline sodium citrate) 0.2% SDS (sodium dodecyl sulphate) at 42° C., followed by washing in 0.1×SSC, and 0.1% SDS at 65° C. Moderate stringency refers to conditions equivalent to hybridization in 50% formamide, 5×Denhardt's solution, 5×SSC, 0.2% SDS at 42° C., followed by washing in 0.2×SSC, 0.2% SDS, at 65° C. Low stringency refers to conditions equivalent to hybridization in 10% formamide, 5×Denhardt's solution, 6×SSC, 0.2% SDS, followed by washing in 1×SSC, 0.2% SDS, at 50° C.

### BRIEF DESCRIPTION OF THE FIGURES

FIG. 1 is a graph depicting the overall survival rate of patients with AML under the age of 50. The time period is approximately 364 weeks. Patients were separated into two groups: 1) AML patients with wildtype WT1 gene (solid line) and 2) AML patients with a mutation in WT1 (dotted line).

FIG. 2 is a graph depicting the overall survival rate of patients with AML under the age of 50 having intermediate cytogenetics. The time period is approximately 364 weeks. Patients were separated into two groups: 1) AML patients with wildtype WT1 gene (solid line) and 2) AML patients with a mutation in WT1 (dotted line).

FIG. 3 is a graph depicting the event free survival rate of patients with AML under the age of 50, over a time period of approximately 364 weeks. Patients were separated into two groups: 1) AML patients with wildtype WT1 gene (solid line) and 2) AML patients with a mutation in WT1 (dotted line).

FIG. 4 is a graph depicting the complete remission duration of patients with AML under the age of 50, over a time period of approximately 364 weeks. Patients were separated into two groups: 1) AML patients with wildtype WT1 gene (solid line) and 2) AML patients with a mutation in WT 1 (dotted line).

FIG. 5 is a graph depicting the survival rate of patients with AML over a time period of approximately 364 weeks. Patients were separated into two groups: 1) patients who have an A/A or A/G genotype at R301 (solid line) and 2) patients who have a G/G genotype at R301 (dotted line).

FIG. 6 is the cDNA sequence of the WT1 gene and is provided as SEQ ID NO: 1.

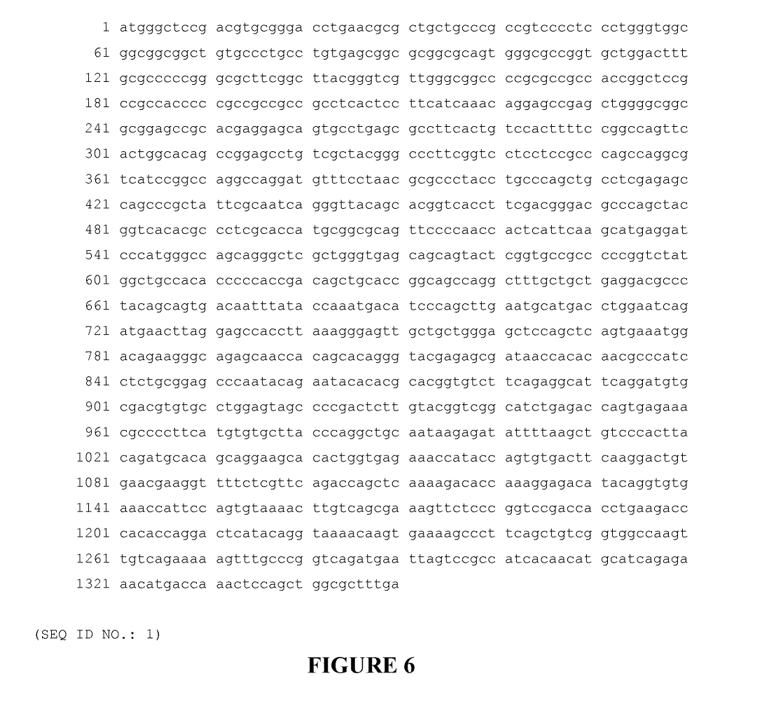

FIG. 7 is the amino acid sequence of the WT1 protein and is provided as SEQ ID NO: 2.

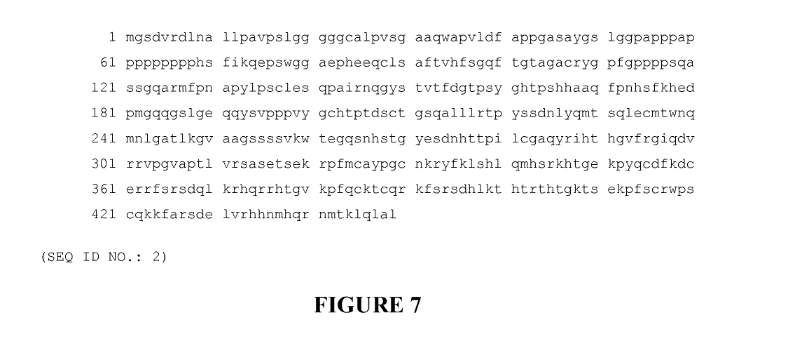

### DETAILED DESCRIPTION

The present invention is based on the identification of several mutations in exon 7 of the WT1 gene, including: two single nucleotide polymorphisms (SNPs), three tandem duplications, a deletion, and two insertion deletions. Exemplary DNA sequence of wildtype WT1 includes but not limited to GenBank accession number AH003034 and is provided as SEQ ID NO: 1. The amino acid sequence of the WT1 protein includes GenBank accession number AAA61299 and is provided as (SEQ ID NO: 2). One SNP is in the WT1 gene codon encoding the arginine at residue 301 of the WT1 protein (“R301”). The codon encoding R301 in SEQ ID NO: 2 corresponds to nucleotides 901-903 of SEQ ID NO: 1, and the SNP is found at position 903, the third nucleotide of the codon. It was found that position 903 was polymorphic for either an adenine or a guanine identified as A903 and G903, respectively. Both polymorphic variants encode arginine and do not result in a mutated protein. Other mutations included: “C938A”, a non-sense SNP from C to A at position 938 of SEQ ID NO: 1 which encodes a stop codon at R313 of SEQ ID NO: 2; “902-939 dup”, a tandem adjacent duplication of the sequence from position 902 through 939 of SEQ ID NO: 1; “929-933 dup”, a tandem adjacent duplication of the sequence from position 929 through 933 of SEQ ID NO: 1; “939-959 dup”, a tandem adjacent duplication of the sequence from position 939 through 959 of SEQ ID NO: 1; “912-917del”, a deletion of nucleotides from position 912 through 917 of SEQ ID NO: 1; “912-917indel14”, a deletion of the nucleotides from position 912 through 917 of SEQ ID NO: 1 and the insertion of SEQ ID NO: 13; and “912-917indel23”, a deletion of the nucleotides from position 912 through 917 of SEQ ID NO: 1 and the insertion of SEQ ID NO: 14.

Patients diagnosed as having a myeloproliferative disorder who were homozygous for the G903 SNP (“the G/G genotype”) had a longer survival time relative to heterozygotes (“the A/G genotype”) or homozygotes having A903 (“the A/A genotype”). The effect of genotype on survival time is particularly evident in patients under 50 years of age. The C938A SNP, the tandem duplications, the deletion, and the insertion-deletions also correlated with shorter overall survival time in patients under 50 years of age. Accordingly, the invention also provides variant nucleic acids or SNPs associated with WT 1 gene mutations, methods and reagents for the detection of the variants disclosed herein, uses of these variants for the development of detection reagents, and assays or kits that utilize such reagents.

### Sample Collection and Preparation

The methods and compositions of this invention may be used to detect polymorphisms in the WT1 gene using a biological sample obtained from an individual. The nucleic acid (DNA or RNA) may be isolated from the sample according to any methods well known to those of skill in the art. Examples include tissue samples or any cell-containing or acellular bodily fluid. Biological samples may be obtained by standard procedures and may be used immediately or stored, under conditions appropriate for the type of biological sample, for later use.

Methods of obtaining test samples are well known to those of skill in the art and include, but are not limited to, aspirations, tissue sections, drawing of blood or other fluids, surgical or needle biopsies, and the like. The test sample may be obtained from an individual or patient diagnosed as having a myeloproliferative disorder or suspected being afflicted with a myeloproliferative disorder. The test sample may be a cell-containing liquid or a tissue. Samples may include, but are not limited to, amniotic fluid, biopsies, blood, blood cells, bone marrow, fine needle biopsy samples, peritoneal fluid, amniotic fluid, plasma, pleural fluid, saliva, semen, serum, tissue or tissue homogenates, frozen or paraffin sections of tissue. Samples may also be processed, such as sectioning of tissues, fractionation, purification, or cellular organelle separation.

If necessary, the sample may be collected or concentrated by centrifugation and the like. The cells of the sample may be subjected to lysis, such as by treatments with enzymes, heat, surfactants, ultrasonication, or a combination thereof. The lysis treatment is performed in order to obtain a sufficient amount of nucleic acid derived from the individual's cells to detect using polymerase chain reaction.

Methods of plasma and serum preparation are well known in the art. Either “fresh” blood plasma or serum, or frozen (stored) and subsequently thawed plasma or serum may be used. Frozen (stored) plasma or serum should optimally be maintained at storage conditions of −20 to −70° C. until thawed and used. “Fresh” plasma or serum should be refrigerated or maintained on ice until used, with nucleic acid (e.g., RNA, DNA or total nucleic acid) extraction being performed as soon as possible. Exemplary methods are described below.

Blood can be drawn by standard methods into a collection tube, typically siliconized glass, either without anticoagulant for preparation of serum, or with EDTA, sodium citrate, heparin, or similar anticoagulants for preparation of plasma. If preparing plasma or serum for storage, although not an absolute requirement, is that plasma or serum is first fractionated from whole blood prior to being frozen. This reduces the burden of extraneous intracellular RNA released from lysis of frozen and thawed cells which might reduce the sensitivity of the amplification assay or interfere with the amplification assay through release of inhibitors to PCR such as porphyrins and hematin. “Fresh” plasma or serum may be fractionated from whole blood by centrifugation, using gentle centrifugation at 300-800 times gravity for five to ten minutes, or fractionated by other standard methods. High centrifugation rates capable of fractionating out apoptotic bodies should be avoided. Since heparin may interfere with RT-PCR, use of heparinized blood may require pretreatment with heparanase, followed by removal of calcium prior to reverse transcription. Imai, H., et al., J. Virol. Methods 36:181-184, (1992). Thus, EDTA is a suitable anticoagulant for blood specimens in which PCR amplification is planned.

### Nucleic Acid Extraction and Amplification

The nucleic acid to be amplified may be from a biological sample such as an organism, cell culture, tissue sample, and the like. The biological sample can be from a subject which includes any animal, preferably a mammal. A preferred subject is a human, which may be a patient presenting to a medical provider for diagnosis or treatment of a disease. The volume of plasma or serum used in the extraction may be varied dependent upon clinical intent, but volumes of 100 μL to one milliliter of plasma or serum are usually sufficient.

Various methods of extraction are suitable for isolating the DNA or RNA. Suitable methods include phenol and chloroform extraction. See Maniatis et al., Molecular Cloning, A Laboratory Manual, 2d, Cold Spring Harbor Laboratory Press, page 16.54 (1989). Numerous commercial kits also yield suitable DNA and RNA including, but not limited to, QIAamp™ mini blood kit, Agencourt Genfind™, Roche Cobas® Roche MagNA Pure® or phenol:chloroform extraction using Eppendorf Phase Lock Gels®, and the NucliSens extraction kit (Biomerieux, Marcy l'Etoile, France). In other methods, mRNA may be extracted from patient blood/bone marrow samples using MagNA Pure LC mRNA HS kit and Mag NA Pure LC Instrument (Roche Diagnostics Corporation, Roche Applied Science, Indianapolis, Ind.).

Nucleic acid extracted from tissues, cells, plasma or serum can be amplified using nucleic acid amplification techniques well know in the art. Many of these amplification methods can also be used to detect the presence of mutations simply by designing oligonucleotide primers or probes to interact with or hybridize to a particular target sequence in a specific manner. By way of example, but not by way of limitation, these techniques can include the polymerase chain reaction (PCR), reverse transcriptase polymerase chain reaction (RT-PCR), nested PCR, ligase chain reaction. See Abravaya, K., et al., Nucleic Acids Research, 23:675-682, (1995), branched DNA signal amplification, Urdea, M. S., et al., AIDS, 7 (suppl 2):S11-S 14, (1993), amplifiable RNA reporters, Q-beta replication, transcription-based amplification, boomerang DNA amplification, strand displacement activation, cycling probe technology, isothermal nucleic acid sequence based amplification (NASBA). See Kievits, T. et al., J Virological Methods, 35:273-286, (1991), Invader Technology, or other sequence replication assays or signal amplification assays. These methods of amplification each described briefly below and are well-known in the art.

Some methods employ reverse transcription of RNA to cDNA. As noted, the method of reverse transcription and amplification may be performed by previously published or recommended procedures, which referenced publications are incorporated herein by reference in their entirety. Various reverse transcriptases may be used, including, but not limited to, MMLV RT, RNase H mutants of MMLV RT such as Superscript and Superscript II (Life Technologies, GIBCO BRL, Gaithersburg, Md.), AMV RT, and thermostable reverse transcriptase from Thermus Thermophilus. For example, one method, but not the only method, which may be used to convert RNA extracted from plasma or serum to cDNA is the protocol adapted from the Superscript II Preamplification system (Life Technologies, GIBCO BRL, Gaithersburg, Md.; catalog no. 18089-011), as described by Rashtchian, A., PCR Methods Applic., 4:S83-S91, (1994).

PCR is a technique for making many copies of a specific template DNA sequence. The reaction consists of multiple amplification cycles and is initiated using a pair of primer sequences that hybridize to the 5′ and 3′ ends of the sequence to be copied. The amplification cycle includes an initial denaturation, and typically up to 50 cycles of annealing, strand elongation and strand separation (denaturation). In each cycle of the reaction, the DNA sequence between the primers is copied. Primers can bind to the copied DNA as well as the original template sequence, so the total number of copies increases exponentially with time. PCR can be performed as according to Whelan, et al., J of Clin Micro, 33(3):556-561 (1995). Briefly, a PCR reaction mixture includes two specific primers, dNTPs, approximately 0.25 U of Taq polymerase, and 1×PCR Buffer.

LCR is a method of DNA amplification similar to PCR, except that it uses four primers instead of two and uses the enzyme ligase to ligate or join two segments of DNA. LCR can be performed as according to Moore et al., J Clin Micro, 36(4):1028-1031 (1998). Briefly, an LCR reaction mixture contains two pair of primers, dNTP, DNA ligase and DNA polymerase representing about 90 μl, to which is added 100 μl of isolated nucleic acid from the target organism. Amplification is performed in a thermal cycler (e.g., LCx of Abbott Labs, Chicago, Ill.).

TAS is a system of nucleic acid amplification in which each cycle is comprised of a cDNA synthesis step and an RNA transcription step. In the cDNA synthesis step, a sequence recognized by a DNA-dependent RNA polymerase (i.e., a polymerase-binding sequence or PBS) is inserted into the cDNA copy downstream of the target or marker sequence to be amplified using a two-domain oligonucleotide primer. In the second step, an RNA polymerase is used to synthesize multiple copies of RNA from the cDNA template. Amplification using TAS requires only a few cycles because DNA-dependent RNA transcription can result in 10-1000 copies for each copy of cDNA template. TAS can be performed according to Kwoh et al., PNAS, 86:1173-7 (1989). Briefly, extracted RNA is combined with TAS amplification buffer and bovine serum albumin, dNTPs, NTPs, and two oligonucleotide primers, one of which contains a PBS. The sample is heated to denature the RNA template and cooled to the primer annealing temperature. Reverse transcriptase (RT) is added the sample incubated at the appropriate temperature to allow cDNA elongation. Subsequently T7 RNA polymerase is added and the sample is incubated at 37° C. for approximately 25 minutes for the synthesis of RNA. The above steps are then repeated. Alternatively, after the initial cDNA synthesis, both RT and RNA polymerase are added following a 1 minute 100° C. denaturation followed by an RNA elongation of approximately 30 minutes at 37° C. TAS can be also be performed on solid phase as according to Wylie et al., J Clin Micro, 36(12):3488-3491 (1998). In this method, nucleic acid targets are captured with magnetic beads containing specific capture primers. The beads with captured targets are washed and pelleted before adding amplification reagents which contains amplification primers, dNTP, NTP, 2500 U of reverse transcriptase and 2500 U of T7 RNA polymerase. A 100 μl TMA reaction mixture is placed in a tube, 200 μl oil reagent is added and amplification is accomplished by incubation at 42° C. in a waterbath for one hour.

NASBA is a transcription-based amplification method which amplifies RNA from either an RNA or DNA target. NASBA is a method used for the continuous amplification of nucleic acids in a single mixture at one temperature. For example, for RNA amplification, avian myeloblastosis virus (AMV) reverse transcriptase, RNase H and T7 RNA polymerase are used. This method can be performed as according to Heim, et al., Nucleic Acids Res., 26(9):2250-2251 (1998). Briefly, an NASBA reaction mixture contains two specific primers, dNTP, NTP, 6.4 U of AMV reverse transcriptase, 0.08 U of Escherichia coli Rnase H, and 32 U of T7 RNA polymerase. The amplification is carried out for 120 min at 41° C. in a total volume of 20 μl.

In a related method, self-sustained sequence-replication (3SR) reaction, isothermal amplification of target DNA or RNA sequences in vitro using three enzymatic activities: reverse transcriptase, DNA-dependent RNA polymerase and Escherichia coli ribonuclease H. This method may be modified from a 3-enzyme system to a 2-enzyme system by using human immunodeficiency virus (HIV)-1 reverse transcriptase instead of avian myeloblastosis virus (AMV) reverse transcriptase to allow amplification with T7 RNA polymerase but without E. coli ribonuclease H. In the 2-enzyme 3SR, the amplified RNA is obtained in a purer form compared with the 3-enzyme 3SR (Gebinoga & Oehlenschlager Eur J Biochem, 235:256-261, 1996).

SDA is an isothermal nucleic acid amplification method. A primer containing a restriction site is annealed to the template. Amplification primers are then annealed to 5′ adjacent sequences (forming a nick) and amplification is started at a fixed temperature. Newly synthesized DNA strands are nicked by a restriction enzyme and the polymerase amplification begins again, displacing the newly synthesized strands. SDA can be performed as according to Walker, et al., PNAS, 89:392-6 (1992). Briefly, an SDA reaction mixture contains four SDA primers, dGTP, dCTP, dTTP, dATP, 150 U of Hinc II, and 5 U of exonuclease-deficient of the large fragment of E. coli DNA polymerase I (exo⁻Klenow polymerase). The sample mixture is heated 95° C. for 4 minutes to denature target DNA prior to addition of the enzymes. After addition of the two enzymes, amplification is carried out for 120 min. at 37° C. in a total volume of 50 μl. Then, the reaction is terminated by heating for 2 min. at 95° C.

The Q-beta replication system uses RNA as a template. Q-beta replicase synthesizes the single-stranded RNA genome of the coliphage Qβ. Cleaving the RNA and ligating in a nucleic acid of interest allows the replication of that sequence when the RNA is replicated by Q-beta replicase (Kramer & Lizardi Trends Biotechnol. 1991 9(2):53-8, 1991).

A variety of amplification enzymes are well known in the art and include, for example, DNA polymerase, RNA polymerase, reverse transcriptase, Q-beta replicase, thermostable DNA and RNA polymerases. Because these and other amplification reactions are catalyzed by enzymes, in a single step assay the nucleic acid releasing reagents and the detection reagents should not be potential inhibitors of amplification enzymes if the ultimate detection is to be amplification based. Amplification methods suitable for use with the present methods include, for example, strand displacement amplification, rolling circle amplification, primer extension preamplification, or degenerate oligonucleotide PCR (DOP). These methods of amplification are well known in the art and each described briefly below.

In suitable embodiments, PCR is used to amplify a target or marker sequence of interest. The skilled artisan is capable of designing and preparing primers that are appropriate for amplifying a target or marker sequence. The length of the amplification primers depends on several factors including the nucleotide sequence identity and the temperature at which these nucleic acids are hybridized or used during in vitro nucleic acid amplification. The considerations necessary to determine a preferred length for an amplification primer of a particular sequence identity are well-known to a person of ordinary skill. For example, the length of a short nucleic acid or oligonucleotide can relate to its hybridization specificity or selectivity.

For analyzing SNPs and other variant nucleic acids, it may be appropriate to use oligonucleotides specific for alternative alleles. Such oligonucleotides which detect single nucleotide variations in target sequences may be referred to by such terms as “allele-specific probes”, or “allele-specific primers”. The design and use of allele-specific probes for analyzing polymorphisms is described in, e.g., Mutation Detection A Practical Approach, ed. Cotton et al. Oxford University Press, 1998; Saiki et al., Nature, 324:163-166 (1986); Dattagupta, EP235,726; and Saiki, WO 89/11548. In one embodiment, a probe or primer may be designed to hybridize to a segment of target DNA such that the SNP aligns with either the 5′ most end or the 3′ most end of the probe or primer.

In some embodiments, the amplification may include a labeled primer, thereby allowing detection of the amplification product of that primer. In particular embodiments, the amplification may include a multiplicity of labeled primers; typically, such primers are distinguishably labeled, allowing the simultaneous detection of multiple amplification products.

In one type of PCR-based assay, an allele-specific primer hybridizes to a region on a target nucleic acid molecule that overlaps a SNP position (e.g., nucleotide position 903 of SEQ ID NO: 1) and only primes amplification of an allelic form to which the primer exhibits perfect complementarity (Gibbs, 1989, Nucleic Acid Res., 17:2427-2448). Typically, the primer's 3′-most nucleotide is aligned with and complementary to the SNP position of the target nucleic acid molecule. This primer is used in conjunction with a second primer that hybridizes at a distal site. Amplification proceeds from the two primers, producing a detectable product that indicates which allelic form is present in the test sample. A control is usually performed with a second pair of primers, one of which shows a single base mismatch at the polymorphic site and the other of which exhibits perfect complementarity to a distal site. The single-base mismatch prevents amplification or substantially reduces amplification efficiency, so that either no detectable product is formed or it is formed in lower amounts or at a slower pace. The method generally works most effectively when the mismatch is at the 3′-most position of the oligonucleotide (i.e., the 3′-most position of the oligonucleotide aligns with the target SNP position) because this position is most destabilizing to elongation from the primer (see, e.g., WO 93/22456). Exemplary allele-specific primer sequences for detecting the A903G polymorphism of the WT1 gene are shown in Table 1 below.

TABLE 1
Exemplary Allele-Specific Primers

| Description | Sequence (5′ to 3′) | SEQ ID NO: |
|---|---|---|
| Forward WT | GGGCTACTCCAGGCACACGTC | SEQ ID NO: 4 |
| Allele- | | |
| Specific | | |
| Primer | | |
| Forward | GGGCTACTCCAGGCACACGTT | SEQ ID NO: 5 |
| Mutant | | |
| Allele- | | |
| Specific | | |
| Primer | | |
| Reverse | CCTCTGCGGAGCCCAAT | SEQ ID NO: 6 |
| Primer | | |

In a specific embodiment, a primer contains a sequence substantially complementary to a segment of a target SNP-containing nucleic acid molecule except that the primer has a mismatched nucleotide in one of the three nucleotide positions at the 3′-most end of the primer, such that the mismatched nucleotide does not base pair with a particular allele at the SNP site. In one embodiment, the mismatched nucleotide in the primer is the second from the last nucleotide at the 3′-most position of the primer. In another embodiment, the mismatched nucleotide in the primer is the last nucleotide at the 3′-most position of the primer.

In one embodiment, primer or probe is labeled with a fluorogenic reporter dye that emits a detectable signal. While a suitable reporter dye is a fluorescent dye, any reporter dye that can be attached to a detection reagent such as an oligonucleotide probe or primer is suitable for use in the invention. Such dyes include, but are not limited to, Acridine, AMCA, BODIPY, Cascade Blue, Cy2, Cy3, Cy5, Cy7, Dabcyl, Edans, Eosin, Erythrosin, Fluorescein, 6-Fam, Tet, Joe, Hex, Oregon Green, Rhodamine, Rhodol Green, Tamra, Rox, and Texas Red.

The present invention also contemplates reagents that do not contain (or that are complementary to) a SNP nucleotide identified herein but that are used to assay one or more SNPs disclosed herein. For example, primers that flank, but do not hybridize directly to a target SNP position provided herein are useful in primer extension reactions in which the primers hybridize to a region adjacent to the target SNP position (i.e., within one or more nucleotides from the target SNP site). During the primer extension reaction, a primer is typically not able to extend past a target SNP site if a particular nucleotide (allele) is present at that target SNP site, and the primer extension product can readily be detected in order to determine which SNP allele is present at the target SNP site. For example, particular ddNTPs are typically used in the primer extension reaction to terminate primer extension once a ddNTP is incorporated into the extension product. Thus, reagents that bind to a nucleic acid molecule in a region adjacent to a SNP site, even though the bound sequences do not necessarily include the SNP site itself, are also encompassed by the present invention.

### Detection of Variant Sequences.

Variant nucleic acids may be amplified prior to detection or may be detected directly during an amplification step (i.e., “real-time” methods). In some embodiments, the target sequence is amplified and the resulting amplicon is detected by electrophoresis. In some embodiments, the specific mutation or variant is detected by sequencing the amplified nucleic acid. In some embodiments, the target sequence is amplified using a labeled primer such that the resulting amplicon is detectably labeled. In some embodiments, the primer is fluorescently labeled.

In one embodiment, detection of a variant nucleic acid, such as a SNP, is performed using the TaqMan® assay, which is also known as the 5′ nuclease assay (U.S. Pat. Nos. 5,210,015 and 5,538,848) or Molecular Beacon probe (U.S. Pat. Nos. 5,118,801 and 5,312,728), or other stemless or linear beacon probe (Livak et al., 1995, PCR Method Appl., 4:357-362; Tyagi et al, 1996, Nature Biotechnology, 14:303-308; Nazarenko et al., 1997, Nucl. Acids Res., 25:2516-2521; U.S. Pat. Nos. 5,866,336 and 6,117,635). The TaqMan® assay detects the accumulation of a specific amplified product during PCR. The TaqMan® assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced.

During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target SNP-containing template which is amplified during PCR, and the probe is designed to hybridize to the target SNP site only if a particular SNP allele is present.

TaqMan® primer and probe sequences can readily be determined using the variant and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the variants of the present invention are useful in diagnostic assays for neurodevelopmental disorders and related pathologies, and can be readily incorporated into a kit format. The present invention also includes modifications of the TaqMan® assay well known in the art such as the use of Molecular Beacon probes (U.S. Pat. Nos. 5,118,801 and 5,312,728) and other variant formats (U.S. Pat. Nos. 5,866,336 and 6,117,635).

In an illustrative embodiment, real time PCR is performed using TaqMan® probes in combination with a suitable amplification/analyzer such as the ABI Prism® 7900HT Sequence Detection System. The ABI PRISM® 7900HT Sequence Detection System is a high-throughput real-time PCR system that detects and quantitates nucleic acid sequences. Briefly, TaqMan® probes specific for the amplified target or marker sequence are included in the PCR amplification reaction. These probes contain a reporter dye at the 5′ end and a quencher dye at the 3′ end. Probes hybridizing to different target or marker sequences are conjugated with a different fluorescent reporter dye. During PCR, the fluorescently labeled probes bind specifically to their respective target or marker sequences; the 5′ nuclease activity of Taq polymerase cleaves the reporter dye from the probe and a fluorescent signal is generated. The increase in fluorescence signal is detected only if the target or marker sequence is complementary to the probe and is amplified during PCR. A mismatch between probe and target greatly reduces the efficiency of probe hybridization and cleavage. The ABI Prism 7700HT or 7900HT Sequence detection System measures the increase in fluorescence during PCR thermal cycling, providing “real time” detection of PCR product accumulation. Real time detection on the ABI Prism 7900HT or 7900HT Sequence Detector monitors fluorescence and calculates Rn during each PCR cycle. The threshold cycle, or Ct value, is the cycle at which fluorescence intersects the threshold value. The threshold value is determined by the sequence detection system software or manually.

Exemplary allele-specific probe sequences for detecting the A903G polymorphism of the WT1 gene in a TaqMan assay are shown in Table 2 below.

TABLE 2
Exemplary Allele-Specific TaqMan ® Probes

| Description | Sequence (5′ to 3′) | SEQ ID NO: |
|---|---|---|
| TaqMan WT | CAGGATGTGCGACGTGTGCC | SEQ ID NO: 7 |
| (G903) | | |
| Allele- | | |
| Specific | | |
| Probe | | |
| TaqMan | CAGGATGTGCAACGTGTGCC | SEQ ID NO: 8 |
| Mutant | | |
| (A903) | | |
| Allele- | | |
| Specific | | |
| Probe | | |

Other methods of probe hybridization detected in real time can be used for detecting amplification a target or marker sequence flanking a tandem repeat region. For example, the commercially available MGB Eclipse™ probes (Epoch Biosciences), which do not rely on a probe degradation can be used. MGB Eclipse™ probes work by a hybridization-triggered fluorescence mechanism. MGB Eclipse™ probes have the Eclipse™ Dark Quencher and the MGB positioned at the 5′-end of the probe. The fluorophore is located on the 3′-end of the probe. When the probe is in solution and not hybridized, the three dimensional conformation brings the quencher into close proximity of the fluorophore, and the fluorescence is quenched. However, when the probe anneals to a target or marker sequence, the probe is unfolded, the quencher is moved from the fluorophore, and the resultant fluorescence can be detected.

Oligonucleotide probes can be designed which are between about 10 and about 100 nucleotides in length and hybridize to the amplified region. Oligonucleotides probes are preferably 12 to 70 nucleotides; more preferably 15-60 nucleotides in length; and most preferably 15-25 nucleotides in length. The probe may be labeled. Amplified fragments may be detected using standard gel electrophoresis methods. For example, in preferred embodiments, amplified fractions are separated on an agarose gel and stained with ethidium bromide by methods known in the art to detect amplified fragments.

Another suitable detection methodology involves the design and use of bipartite primer/probe combinations such as Scorpion™ probes. These probes perform sequence-specific priming and PCR product detection is achieved using a single molecule. Scorpion™ probes comprise a 3′ primer with a 5′ extended probe tail comprising a hairpin structure which possesses a fluorophore/quencher pair. The probe tail is “protected” from replication in the 5′ to 3′ direction by the inclusion of hexethlyene glycol (HEG) which blocks the polymerase from replicating the probe. The fluorophore is attached to the 5′ end and is quenched by a moiety coupled to the 3′ end. After extension of the Scorpion™ primer, the specific probe sequence is able to bind to its complement within the extended amplicon thus opening up the hairpin loop. This prevents the fluorescence from being quenched and a signal is observed. A specific target is amplified by the reverse primer and the primer portion of the Scorpion™, resulting in an extension product. A fluorescent signal is generated due to the separation of the fluorophore from the quencher resulting from the binding of the probe element of the Scorpion™ to the extension product. Such probes are described in Whitcombe et al., Nature Biotech 17: 804-807 (1999).

### Determining Prognosis

Provided herein are methods of using the SNP/genotype status at nucleotide position 903 of the WT1 gene in a test sample from a patient, alone or in conjunction with clinical factors, in determining the prognosis for a patient having a myeloproliferative disease. In some embodiments, prognosis may be a prediction of the likelihood that a patient will survive for a particular period of time, or the prognosis is a prediction of how long a patient may live, or the prognosis is the likelihood that a patent will recover from a disease or disorder. There are many ways that prognosis can be expressed. For example prognosis can be expressed in terms of complete remission rates (CR), overall survival (OS) which is the amount of time from entry to death, remission duration, which is the amount of time from remission to relapse or death.

In certain embodiments the 903 SNP status (i.e., A/A, A/G, or G/G) is used as an indicator of an prognosis, for example, in ALL. For example, patients having the G/G genotype (i.e., homozygous for G903) are identified as likely to have a longer survival time relative to those having the A/G or A/A genotypes.

In certain embodiments, the prognosis of ALL, AML, AUL, CLL or CML patients can be correlated to the clinical outcome of the disease using the WT1 genotype status and other clinical factors. Simple algorithms have been described and are readily adapted to this end. The approach by Giles et al., British Journal of Haematology, 121:578-585, is exemplary. As in Giles et al., associations between categorical variables (e.g., proteasome activity levels and clinical characteristics) can be assessed via crosstabulation and Fisher's exact test. Unadjusted survival probabilities can be estimated using the method of Kaplan and Meier. The Cox proportional hazards regression model also can be used to assess the ability of patient characteristics (such as proteasome activity levels) to predict survival, with ‘goodness of fit’ assessed by the Grambsch-Therneau test, Schoenfeld residual plots, martingale residual plots and likelihood ratio statistics (see Grambsch, 1995; Grambsch et al, 1995).

In some embodiments of the invention, multiple prognostic factors, including the WT1 genotype status, are considered when determining the prognosis of a patient. For example, the prognosis of an AML or ALL patient may be determined based on WT1 genotype and one or more prognostic factors selected from the group consisting of cytogenetics, performance status, AHD (antecedent hematological disease), and age. In certain embodiments, other prognostic factors may be combined with the WT1 genotype in the algorithm to determine prognosis with greater accuracy.

### Kits

The invention also provides kits that can be used to assess the WT1 genotype. Thus, in one embodiment, the invention provides a kit for identifying a nucleotide occurrence at a position corresponding to nucleotide 903 of SEQ ID NO: 1. In another embodiment, the invention provides a kit for identifying WT1 mutations selected from the group consisting of: 902-939 dup, 929-933 dup, 939-959 dup, C938A, 912-917 del, 912-917indel14, and 912-917indel23. The kit can include an isolated primer, primer pair, probe, or other specific binding pair member of the present invention, or a combination thereof. The kit can further include reagents for amplifying a polynucleotide using a primer pair. Furthermore, the reagents can include at least one detectable label, which can be used to label the isolated oligonucleotide probe, primer, primer pair, or other specific binding pair member, or can be incorporated into a product generated using the isolated oligonucleotide probe, primer, primer pair, or specific binding pair member. The kit may further include at least one polymerase, ligase, or endonuclease, or a combination thereof. The kit may further include at least one polynucleotide corresponding to a portion of an WT1 gene encompassing nucleotide 903 of SEQ ID NO: 1. This polynucleotide may be used as an allele-specific primer or an allele-specific probe and optionally may be detectably labeled or have one member of a binding pair suitable for attachment of a detectable label. Alternatively, the primer pair may be designed to flank nucleotide 903 in order to amplify (e.g., by PCR) a target region for detection.

In addition, a kit of the invention can contain, for example, reagents for performing a method of the invention, including, for example, one or more detectable labels, which can be used to label a probe or primer or can be incorporated into a product generated using the probe or primer (e.g., an amplification product); one or more polymerases, which can be useful for a method that includes a primer extension or amplification procedure, or other enzyme or enzymes (e.g., a ligase or an endonuclease), which can be useful for performing an oligonucleotide ligation assay or a mismatch cleavage assay; and/or one or more buffers or other reagents that are necessary to or can facilitate performing a method of the invention. The primers or probes can be included in a kit in a labeled form, for example with a label such as biotin.

In another embodiment, the kit contains one or more primers suitable for a single nucleotide primer extension reaction, and one or more chain-terminating labeled nucleotides (e.g., ddNTPs). Primer of type preferably comprise the nucleotide sequence of SEQ ID NO: 4 or 5.

The kit can also include instructions for using the probes or primers to perform a method of the present invention.

### Example 1

### WT1 SNP Polymorphism in Patients with Adult Acute Lymphoblastic Leukemia

### Sample Collection

Peripheral blood samples and bone marrow samples were collected in EDTA-containing tubes (Becton Dickinson, NJ) from 174 newly diagnosed AML patients. Blood or bone marrow cells were separated from plasma by differential centrifugation using Puregene® RBC lysis solution (Gentra System, MN, USA). The cell pellet was washed with phosphate-buffered saline. Both plasma and cell samples were cryopreserved at −80° C. for future use.

### WT1 Mutation Analysis

WT1 mutations in exons 7 and 9 from patient samples were detected by sequencing and fragment length analysis. The findings were correlated with outcome and other laboratory findings.

WT1 mutations (non-sense mutations, duplications, insertions, and deletions) were detected in 14% of patients younger than 50 years of age (n=50) and in 4% of patients older than 70 (n=124). WT1 mutations correlated with higher white cell count (P=0.01) and higher percentage of blasts in bone marrow (0.03) and peripheral blood (P=0.009). In addition there was significant correlation between the presence of WT1 mutation and FLT3 mutation (P=0.002), but not NPM1 mutation (P=0.8). There was significant correlation between WT1 mutation and shorter survival (P=0.025) and event-free survival (P=0.002) as well as shorter complete remission duration (P=0.002) in patients younger than 50. Shorter survival was also demonstrated when only patients with intermediate cytogenetics were considered (P=0.03). There was no correlation between WT mutation and response to therapy.

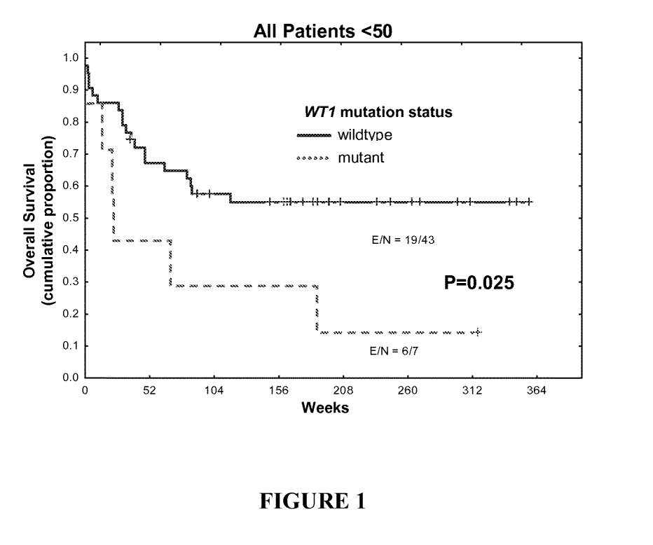

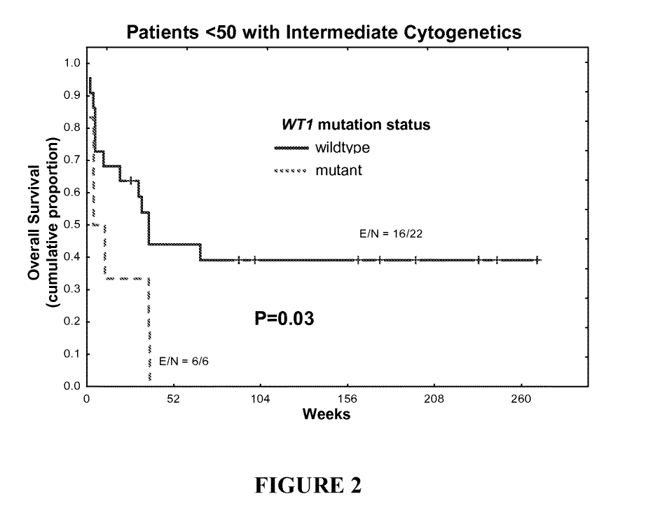

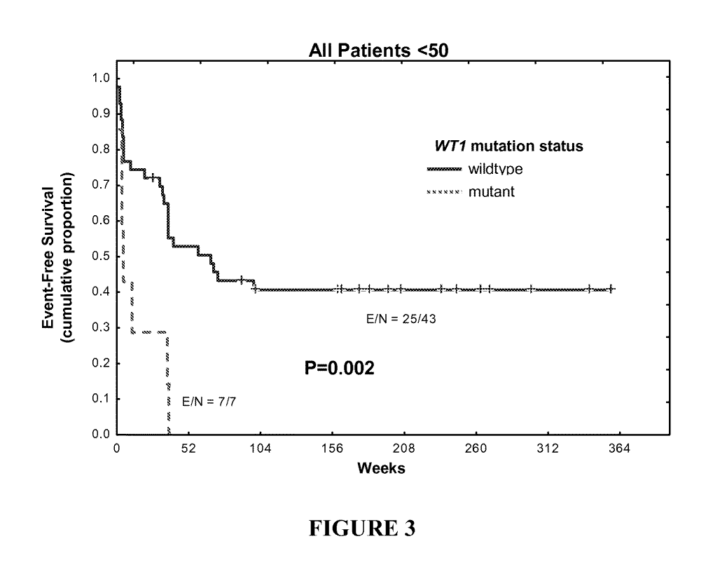

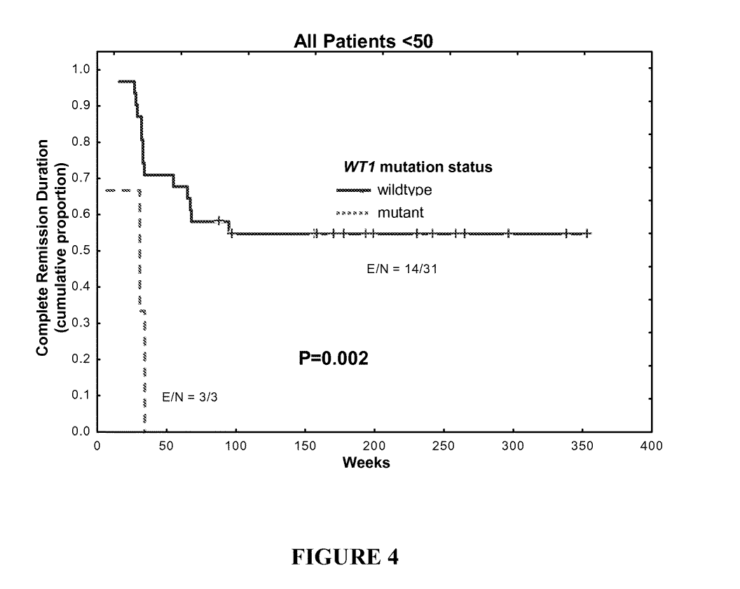

WT1 mutations were significantly associated with shorter overall survival in patients <50 years of age (P=0.025, FIG. 1). This statistically significant association persisted when considering only patients <50 with intermediate cytogenetics (P=0.03, FIG. 2). WT1 mutation was also associated with shorter event-free survival (“EFS”; P=0.002, FIG. 3) and shorter complete remission duration (“CRD”; P=0.002, FIG. 4) in patients <50 years of age.

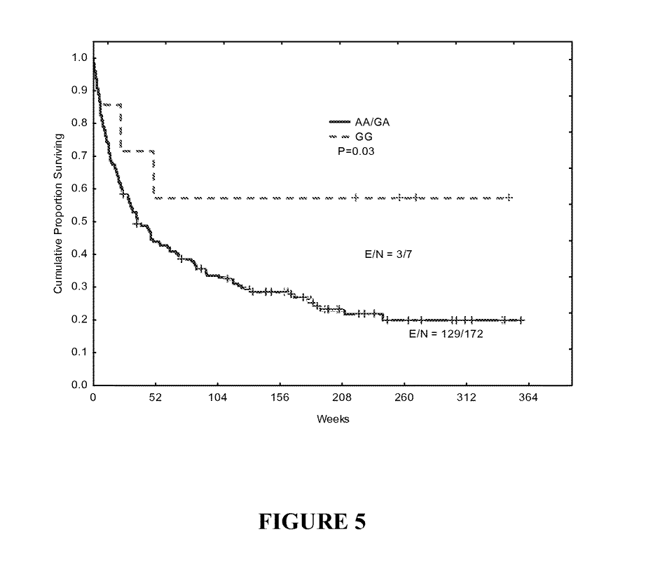

A silent polymorphism A903G was also detected, with A/A, A/G, and G/G genotypes at 70%, 26%, and 4% in these AML patients. Similar distribution of genotypes was detected in normal controls. Significant correlation was observed between the presence of the G/G genotype at R301 and longer survival, irrespective of age (FIG. 5). Both heterozygous A/G and homozygous A/A had shorter survival compared with patients homozygous for the minor allele (G/G).

### Example 2

### WT1 Exon 7 SNP and Exon 9 Polymorphism Detection Using PCR and Sequencing

### Materials and Methods

Peripheral blood and bone marrow samples were collected from a total of 343 patients using the methods described in Example 1 above. Within this group, 93 patients were known to have or suspected of having AML and 250 patient had no known diagnosis of AML. Genomic DNA from peripheral blood cell or bone marrow samples was extracted using Qiagen BioRobot EZ1. Nucleic acid from blood plasma samples was extracted using the NucliSens extraction kit.

WT1 exon 7 SNPs and exon 9 polymorphisms were assessed by amplifying in two separate PCR reactions using a primer pair for exon 7 (WT1-7F and WT1-7R) and a primer pair for exon 9 (WT1-9F and WT1-9R). Each of the primers has an M13 sequencing tag on its 5′ end (noted in lowercase) to allow for sequencing verification after amplification. WT 1-7F (SEQ ID NO: 9), WT1-7R (SEQ ID NO: 10), WT1-9F (SEQ ID NO: 11), and WT1-9R (SEQ ID NO: 12) are referenced below.

SEQ ID NO: 9
tgtaaaacgacggccagtTCTCCCTCAAGACCTACGTGA
SEQ ID NO: 10
tgtaaaacgacggccagtGTGTGAGAGCCTGGAAAAGG
SEQ ID NO: 11
tgtaaaacgacggccagtTCACTGTGCCCACATTGTTAG
SEQ ID NO: 12
tgtaaaacgacggccagtTCCAATCCCTCTCATCACAA

Master mix containing enzymes, buffers, primers and dNTPs was prepared according to table 3 below. Separate master mixes were created for the exon 7 primer pair and the exon 9 primer pair.

TABLE 3
Master Mix Composition

| Components | Final Concentration | |
|---|---|---|
| 10x Reaction Buffer | 1x (2 mM MgCl₂) | |
| With 20 mM MgCl₂ | | |
| dNTP | 250 | μM |
| Forward primer | 0.4 | μM |
| Reverse primer | 0.4 | μM |
| FastStart Taq | 1.25 | U |

DNA template from each sample was added to a portion of the exon 7 and exon 9 master mixes and the separate reaction mixtures was amplified in a thermocycler for 40 cycles (30 seconds at 95° C., then 30 seconds at 60° C., then 1 minute at 72° C.). For each assay batch, a Wilms Tumor1 gene positive control and a reagent-only negative control was included. Amplified product was verified using gel electrophoresis and then purified using a Millipore Multiscreen Separation System.

Purified amplification product was then sequenced by re-amplifying the amplified product using a BigDye Terminator Sequencing kit and then sequencing the reamplified product with an ABI 3730 DNA Analyzer.

### Sequence Analysis Results

Exon 7 mutations were observed in nine samples from the 93 known or suspected AML patient samples. Exon 9 mutations were not found in any samples tested. Three tandem duplication mutations were identified in which the duplicated sequence immediately followed and was adjacent to the native sequence. The tandem duplication mutants were 902-939 dup, 929-933 dup, and 939-959 dup. One single nucleotide polymorphism (SNP) was observed, C938A, which resulted in a stop codon at amino acid position 313. Two insertion-deletion mutations (“indels”) were observed. In each indel, the inserted sequences consisted of full or partial TATA repeats. For convenience the indels are named such that the deleted nucleotides are first identified and the terminal number refers to the number of inserted nucleotides. The two indels were 912-917indel14 and 912-917indel23. Thus, for 912-917indel14, the inserted sequence is TATATATATATATA (SEQ ID NO: 13) and for 912-917indel23 the inserted sequence is TATATATATATATATATATATAT (SEQ ID NO: 14).

A903G, was also examined in the 343 patient samples. Wild type homozygous A/A genotype was found in 70.55% of the patients, heterozygous G/A genotype (A903G) was found in 26.53% of the patients, and homozygous G/G genotype was found in 2.92% of the patients, which is similar to the results of the initial study provided in Example 1. A non-patient population of 27 individuals was also genotyped for the A903G polymorphism in order to obtain a reference range. Patient numbers and their percentages are summarized in Table 4 below.

TABLE 4
Summary of WT1 Exon 7 SNP in patient and normal populations

| | Patients | | Normals | |
|---|---|---|---|---|
| Polymorphic Type | Number | Percentage | Number | Percentage |
| 903A (wild type) | 242 | 70.55% | 18 | 66.67% |
| 903A > R | 91 | 26.53% | 8 | 29.53% |
| 903A > G | 10 | 2.92% | 1 | 3.70% |
| TOTAL (patients) | 343 | — | 27 | — |

### Example 3

### WT1 Exon 7 Mutant Detection Using Fragment Analysis

### Materials and Methods

Peripheral blood and bone marrow samples were collected from the 343 patients of Example 2, using the procedure as described in Example 1 above. Genomic DNA from patient samples was extracted as described in Example 2.

PCR using labeled primer pair WT1-7F and WT1-7R were used to amplify and heterozygous mutations to exon 7 of WT1. WT1-7F has an M13 sequencing tag on its 5′ end (noted in lowercase) to allow for sequencing verification after fragment analysis. WT1-7R was labeled with 6FAM fluorescent label at its 5′ end. WT1-7F is provided below as SEQ ID NO: 15; WT1-7R is provided below as SEQ ID NO: 16.

SEQ ID NO: 15
tgtaaaacgacggccagtTCTCCCTCAAGACCTACGTGA
SEQ ID NO: 16
GTGTGAGAGCCTGGAAAAGG

Master mix containing enzymes, buffers, primers and dNTPs was prepared according to Table 3 of Example 2.

DNA template from each sample was added to the master mix and the reaction mixture was amplified in a thermocycler for 40 cycles (30 seconds at 95° C., then 30 seconds at 60° C., then 1 minute at 72° C.). For each assay batch, a WT1 heterozygous indel mutation positive control and a reagent-only negative control were included.

Amplified product fragment sizes were analyzed using an ABI 3100 Genetic Analyzer. Amplified product was then diluted 1:100 and added in a 1:10 ratio with a loading mix for capillary electrophoresis. Loading mix was HiDi Formamide with GeneScan 350 ROX Size standard at a ratio of 10:0.5. Electropherograms produced from the fragment analysis were processed using Genemapper software.

### Fragment Analysis Results

Wild type samples display only a single peak at 304 base pairs. 8 samples showed one or two peaks in addition to the 304 bp wild type peak which indicated additional amplified fragments between 298 bp and 342 bp. The patient samples and the respective fragment sizes are listed in Table 6 below. The base pairs refer to the number of base pairs above and below wild type 304 bp fragments.

TABLE 6
Mutant Fragments Found by Fragment Analysis

| | | | | Sequenced |
|---|---|---|---|---|
| Sample ID | Fragment 1 | Fragment 2 | Fragment 3 | Mutant |
| A | +20 bp | — | — | 939-959 dup. |
| B | +5 bp | — | — | 929-933 dup. |
| C | −6 bp | +8 bp | +17 bp | 912-917 del., |
| | | | | 912-917indel14, |
| | | | | 912-917indel23 |
| D | +38 bp | — | — | 902-939 dup. |
| E | +38 bp | — | — | 902-939 dup. |
| F | +38 bp | — | — | 902-939 dup. |

The nine samples with mutant exon 7 fragments corresponded to the nine samples detected using PCR sequencing, as described in Example 2.

Unless otherwise defined, all technical and scientific terms used herein have the same meaning as commonly understood by one of ordinary skill in the art to which this invention belongs. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other embodiments are set forth within the following claims.

1. A method of determining a prognosis for a subject diagnosed as having a myeloproliferative disease comprising:
a) determining the zygosity status of the subject for a single nucleotide polymorphism in the WT1 gene at the nucleotide corresponding to position 903 of SEQ ID NO: 1;
b) identifying the subject as having a poor prognosis when the subject has the A/A or A/G phenotype at the nucleotide corresponding to position 903 of SEQ ID NO: 1.

2. A method of determining a prognosis for a subject diagnosed as having a myeloproliferative disease comprising:
a) determining the zygosity status of the subject for at least one mutation in the WT1 gene, wherein the mutation is selected from the group consisting of 902-939 dup, 929-933 dup, 939-959 dup, C938A, 912-917 del, 912-917indel14, and 912-917indel23; and
b) identifying the subject as having a poor prognosis when the subject is heterozygous or homozygous for the mutation.

3. The method of claim 2, wherein the mutation is 902-939 dup, 929-933 dup, or 939-959 dup.

4. The method of claim 2, wherein the mutation is C938A.

5. The method of claim 2, wherein the mutation is 912-917 del, 912-917indel14, or 912-917indel23.

6. The method of claim 1, wherein the myeloproliferative disease is selected from the group consisting of: chronic lymphocytic leukemia, acute lymphoblastic leukemia, acute myelogenous leukemia, chronic myelogenous leukemia, hairy cell leukemia, T-cell prolymphocytic leukemia, large granular lymphocytic leukemia.

7. The method of claim 6, wherein the myeloproliferative disease is acute myelogenous leukemia.

8. The method of claim 1, wherein the zygosity status is determined by assessing subject nucleic acid obtained from a biological sample.

9. The method of claim 8, wherein the biological sample is blood, serum, or plasma.

10. The method of claim 1, wherein the poor prognosis is selected from the group consisting of shorter survival, shorter complete remission duration, and shorter event-free survival.

11. The method of claim 10, wherein the poor prognosis is shorter survival.

12. The method of claim 1, further comprising assessing clinical factors and using the zygosity status and the clinical factors for determining the prognosis.

13. The method of claim 12, wherein at least one of the clinical factors is selected from the group consisting of cytogenetics, peripheral white blood cell count, percentage of blast cells in bone marrow, and percentage of blast cells in blood.

14. The method of claim 13, wherein at least one of the clinical factors is cytogenetics.

15. The method of claim 1, wherein the subject is less than 50 years of age.

16. The method of claim 1, further comprising assessing the presence or absence of a FLT3 mutation and identifying the subject as having a poor prognosis when a FLT3 mutation is present.

17. The method of claim 1, wherein the zygosity status is determined using a technique selected from the group consisting of nucleic acid sequencing, probe hybridization, and a primer extension reaction.

18. A method of determining the myeloproliferative disease status of a subject comprising:
a) determining the zygosity status of the subject for at least one of the mutations of the WT1 gene selected from the group consisting of 902-939 dup, 929-933 dup, 939-959 dup, C938A, 912-917 del, 912-917indel14, and 912-917indel23; and
b) identifying the subject
i) as having a myeloproliferative disease when the subject is homozygous for one of said WT1 mutations
ii) as being predisposed to a myeloproliferative disease when the subject is heterozygous for one of said WT1 mutations, or
iii) as having no predisposition to a myeloproliferative disease caused by one of said WT1 mutations when said WT1 mutations are absent from both alleles of the WT1 gene.

19. The method of claim 18, wherein the mutation is 902-939 dup, 929-933 dup, or 939-959 dup.

20. The method of claim 18, wherein the mutation is C938A.

21. The method of claim 18, wherein the mutation is 912-917 del, 912-917indel14, or 912-917indel23.

22. The method of claim 1, wherein the myeloproliferative disease is selected from the group consisting of: chronic lymphocytic leukemia, acute lymphoblastic leukemia, acute myelogenous leukemia, chronic myelogenous leukemia, hairy cell leukemia, T-cell prolymphocytic leukemia, large granular lymphocytic leukemia.

23. The method of claim 22, wherein the myeloproliferative disease is acute myelogenous leukemia.

24. The method of claim 1, wherein the zygosity status is determined by assessing subject nucleic acid obtained from a biological sample.

25. The method of claim 24, wherein the biological sample is blood, serum, or plasma.

26. The method of claim 1, further comprising assessing clinical factors and using the zygosity status and the clinical factors for determining the diagnosis.

27. The method of claim 26, wherein at least one of the clinical factors is selected from the group consisting of cytogenetics, peripheral white blood cell count, percentage of blast cells in bone marrow, and percentage of blast cells in blood.

28. The method of claim 27, wherein at least one of the clinical factors is cytogenetics.

29. The method of claim 1, wherein the subject is less than 50 years of age.

30. The method of claim 1, further comprising assessing the presence or absence of a FLT3 mutation and identifying the subject as having a myeloproliferative disease when a FLT3 mutation is present.

31. The method of claim 1, wherein the zygosity status is determined using a technique selected from the group consisting of nucleic acid sequencing, probe hybridization, and a primer extension reaction.


SEQUENCE LISTING (16 sequences)

SEQ ID NO: 1 - DNA, 1350 bp, Homo sapiens
atgggctccgacgtgcgggacctgaacgcgctgctgcccgccgtcccctccctgggtggcggcggcggctgtgccctgcctgtgagcggcgcggcgcagtgggcgccggtgctggactttgcgcccccgggcgcttcggcttacgggtcgttgggcggccccgcgccgccaccggctccgccgccacccccgccgccgccgcctcactccttcatcaaacaggagccgagctggggcggcgcggagccgcacgaggagcagtgcctgagcgccttcactgtccacttttccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggagttgctgctgggagctccagctcagtgaaatggacagaagggcagagcaaccacagcacagggtacgagagcgataaccacacaacgcccatcctctgcggagcccaatacagaatacacacgcacggtgtcttcagaggcattcaggatgtgcgacgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtgagaaaccataccagtgtgacttcaaggactgtgaacgaaggttttctcgttcagaccagctcaaaagacaccaaaggagacatacaggtgtgaaaccattccagtgtaaaacttgtcagcgaaagttctcccggtccgaccacctgaagacccacaccaggactcatacaggtaaaacaagtgaaaagcccttcagctgtcggtggccaagttgtcagaaaaagtttgcccggtcagatgaattagtccgccatcacaacatgcatcagagaaacatgaccaaactccagctggcgctttga

SEQ ID NO: 2 - protein, 449 aa, Homo sapiens
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 3 - skipped

SEQ ID NO: 4 - DNA, 21 bp, Artificial Sequence
  Description of Artificial Sequence: Synthetic primer
gggctactccaggcacacgtc

SEQ ID NO: 5 - DNA, 21 bp, Artificial Sequence
  Description of Artificial Sequence: Synthetic primer
gggctactccaggcacacgtt

SEQ ID NO: 6 - DNA, 17 bp, Artificial Sequence
  Description of Artificial Sequence: Synthetic primer
cctctgcggagcccaat

SEQ ID NO: 7 - DNA, 20 bp, Artificial Sequence
  Description of Artificial Sequence: Synthetic probe
caggatgtgcgacgtgtgcc

SEQ ID NO: 8 - DNA, 20 bp, Artificial Sequence
  Description of Artificial Sequence: Synthetic probe
caggatgtgcaacgtgtgcc

SEQ ID NO: 9 - DNA, 39 bp, Artificial Sequence
  Description of Artificial Sequence: Synthetic primer
tgtaaaacgacggccagttctccctcaagacctacgtga

SEQ ID NO: 10 - DNA, 38 bp, Artificial Sequence
  Description of Artificial Sequence: Synthetic primer
tgtaaaacgacggccagtgtgtgagagcctggaaaagg

SEQ ID NO: 11 - DNA, 39 bp, Artificial Sequence
  Description of Artificial Sequence: Synthetic primer
tgtaaaacgacggccagttcactgtgcccacattgttag

SEQ ID NO: 12 - DNA, 38 bp, Artificial Sequence
  Description of Artificial Sequence: Synthetic primer
tgtaaaacgacggccagttccaatccctctcatcacaa

SEQ ID NO: 13 - DNA, 14 bp, Artificial Sequence
  Description of Artificial Sequence: Synthetic oligonucleotide
tatatatatatata

SEQ ID NO: 14 - DNA, 23 bp, Artificial Sequence
  Description of Artificial Sequence: Synthetic oligonucleotide
tatatatatatatatatatatat

SEQ ID NO: 15 - DNA, 39 bp, Artificial Sequence; identical to SEQ ID NO: 9
  Description of Artificial Sequence: Synthetic primer

SEQ ID NO: 16 - DNA, 20 bp, Artificial Sequence
  Description of Artificial Sequence: Synthetic primer
gtgtgagagcctggaaaagg